(19) United States
(12) Patent Application Publication
(10) Pub. No.: US 2011/0054413 A1
(43) Pub. Date: Mar. 3, 2011

(54) Pin for hygienic and/or medical use in the anal region

(76) Inventors: Ludwig Römhild, Bischofswiesen (DE); Hannes Römhild, Berlin (DE)

(21) Appl. No.: 12/991,430

(22) PCT Filed: Apr. 24, 2009
(86) PCT No.: PCT/EP09/54968

§ 371 Date: Nov. 6, 2010

(30) Foreign Application Priority Data
May 6, 2008 (DE) 10 2008 026 972.2

(51) Int. Cl. A61M 31/00; A61M 23/00

(52) U.S. Cl. 604/212; 604/540; 604/48

(57) ABSTRACT

A pin for hygienic and/or medical use in the anal region includes a first section extending from a predetermined end of the pin in the longitudinal direction thereof, and a second section that is offset from the first section in the longitudinal direction of the pin. The first section is introduced into the anal region of a patient and the second section enables a user to hold the pin. The first section includes, on its surface, one or more receiving elements, by way of which stool remainders can be removed from the anal region and/or substances can be supplied to the anal region.

### TECHNICAL FIELD

The disclosed subject matter relates to a pin for hygienic and/or medical use in the anal region.

### BACKGROUND

The active cleaning of the anal region has always been limited essentially to the outer anal region, i.e., the region outside the sphincter of the anus. Even for proctologic diseases, an active reduction of food remainders and bacteria by the patient within the inner anal region behind the sphincter, formed by the anal canal, is assigned small significance.

### SUMMARY

A pin is described for cleaning the anal region. The pin includes at least a first section extending from a predetermined end of the pin in its longitudinal direction, which is introducible into the anal region of a patient for performing a hygienic and/or medical application. The term “longitudinal direction” is thereby to be broadly understood. In particular, the longitudinal direction does not need to extend necessarily straight, but it can under some circumstances also be curved, as long as the pin has a curved form. The pin also includes a second section that enables the pin to be held by a user for performing the hygienic and/or medical application, the second section being displaced in longitudinal direction from the first section. Thereby, the user can be a physician or medical personnel. Preferably, however, the pin is used by the patient. One or more receiving elements are formed on the surface of the first section with which stool remainders can be removed from the anal region and/or substances can be supplied to the anal region. Substances include, in particular, liquids such as, e.g. water or oils, whereby the respective liquids, where appropriate, can also be enriched with active ingredients for the respective treatment of the anal region.

The pin is characterized in that the first section is configured such that it can be inserted into the anal region of a patient without injury, whereby via the receiving elements, the removal of stool remainders out of the anal region and/or the introduction of substances in the anal region is made possible in a simple manner.

In some embodiments of the pin, at least one limiting element is provided between the first and the second section, by which the insertion of the pin is limited to a maximum depth in the anal region. In this manner, it is ensured that the pin is not inserted by the user too deeply, and, in particular, also not completely into the rectum, thereby preventing injury. The at least one limiting element includes preferably a plate-shaped intermediary part, extending essentially orthogonally to the longitudinal direction of the pin. By suitable dimensioning of that plate-shaped intermediary part, an insertion into the anal region that is too deep becomes efficiently prevented. The term “plate-shaped” is to be understood broadly and comprises along with round forms, where appropriate, also arbitrary other forms like, e.g., polygons, ellipses, etc. Preferably, the intermediary part includes a maximum extension in direction orthogonally to the longitudinal direction of the pin that is between 20 mm and 35 mm, preferably between 25 mm and 30 mm.

In one application, the pin can be used for the cleaning of the anal canal behind the patient's sphincter. In this case, the first section has a maximum diameter such that it is insertable into the patient's anal canal being behind the anus. In particular, the maximum diameter should be between 8 mm and 25 mm, preferably between 8 mm and 20 mm or between 20 mm and 25 mm. For patients without incontinence problems, a maximum diameter, in particular, between 8 mm and 20 mm is sufficient. For patients with incontinence problems, i.e., with a leaking sphincter, the maximum diameter should be selected to be bigger and rather be in the range between 20 mm and 25 mm.

The embodiment of the pin as a cleaning pin for the anal canal makes possible an advanced cleaning of the inner anal region behind the anus in an easy, inexpensive, and straightforward manner. In particular, an accumulation of stool remainders behind the sphincter can be reduced substantially. Furthermore, a blockage or a cover of numerous different glands, fissures, mucosa flaps, and crypts can be reduced. Likewise, inflammations and irritations can be positively affected. Beyond that, soothing and/or therapeutic substances can be supplied in a straightforward manner behind the sphincter in the inner anal region. This embodiment of the cleaning pin has, besides a cleaning effect, a medical effect, by preventing prophylactically a plurality of diseases in the anal canal and around the sphincter.

Where appropriate, the pin can also be configured such that it serves only for the cleaning of the anal region before the anus. In this case, the maximum diameter of the first section is dimensioned in such a manner that the first section is not insertable beyond the anus into the anal region of a patient. “Not insertable” means that an insertion of the pin into the anal canal is not possible or possible only by force and pain or with injuries possible. Preferably, the maximum diameter is then larger than or equal to 30 mm. To suitably ensure the first section to reach guaranteed the anal region, the maximum diameter should, however, also be not selected too large. In a preferred embodiment, the maximum diameter is therefore smaller than or equal to 40 mm. The main purpose of that embodiment is the hygienic cleaning of the anus without contamination of the hands after the bowel movement. However, also with that embodiment, inflammations caused by stool remainders within the outer anal region in front of the sphincter can be prevented prophylactically.

In a further embodiment, the surface of the first section has a basic form being rotationally symmetric regarding the longitudinal direction of the pin, in particular a conical and/or cylindrical and/or wavy basic form. The basic form is thereby understood as the form of the surface without consideration of the receiving elements, i.e., essentially the envelope surface of the first section.

In order to efficiently avoid injuries within the anal region and, in particular, also in the anal canal behind the sphincter, at least the surface of the first section is burr-free in an embodiment. Likewise, injuries can be prevented by a smooth surface, which has an averaged roughness depth of 4 μm or less.

To allow a simple insertion of the pin into the anal region, the pin is rounded at the predetermined end, for example, in the form of a mushroom or a strawberry or the slender end of a popsicle.

In some embodiments, the one or more receiving elements have a number of steps, whereby a respective step is formed as an annular recess extending in circumferential direction around the pin or as an helical recess extending along the surface of the first section, whereby the recess is preferably arranged between two adjacent axial positions, i.e., the positions being offset with respect to each other in longitudinal direction of the pin, whereby an axial position is arranged, in particular, on a cylindrical partial section of the first section. The length of the recess in longitudinal direction of the pin preferably lies between 2 mm and 10 mm, whereby an effective picking-up of stool remainders or an effective introducing of substances into the anal region is achieved. To avoid injuries in the anal region, the recess is preferably rounded at its edges, whereby the edges have, in particular, a radius of at least 0.2 mm, in particular between 0.2 mm and 1 mm and particularly preferred of 0.5 mm.

In a further embodiment, the cross section of the recess extends in direction away from the predetermined end of the pin from a cut-in at an axial position along a rising slope to an adjacent axial position. In this way, a pin is provided, with which stool remainders can efficiently be removed from the anal region. Likewise, the possibility exists that the cross section of the recess towards the predetermined end of the pin extends from a cut-in at an axial position along a rising slope to an adjacent axial position. In that case, the pin is suitable, in particular, for introducing substances, such as, e.g., oils or liquids, into the anal region.

For effective cleaning of the anal region and/or introducing substances, in particular, recesses which have a maximum depth between 1 mm and 5 mm, particularly, between 1 mm and 3 mm or between 2 mm and 5 mm, proved reasonable. The depth within the range of 1 mm to 3 mm is, in particular, used with pins, which are intended for patients without incontinence problems. The depth between 2 mm and 5 mm is preferably selected when the pin is intended for patients with incontinence problems.

For the above described step-shaped embodiment of the one or more receiving elements, there is further the possibility that in direction away from the predetermined end, the diameter of the first section decreases, and/or increases, and/or remains constant at axial positions. The decrease of the diameters is, in particular, used with such embodiments of the pin, with which stool remainders are to be removed from the anal region, whereas the embodiment with increasing diameter is preferably used with those embodiments of the pin, with which substances should be applied in the anal region.

In some embodiments of the pin, the diameter decreases at two adjacent axial positions by a value, which is between 0.5 mm and 2 mm. This value can remain constant over the axial positions, or this value can, however, also change, for example, the decrease of the diameter can become ever smaller and also larger for axial positions being further and further away from the predetermined end.

In a further embodiment, the one or more receiving elements include a number of troughs and/or grooves, in particular, annular and/or helical grooves extending at the surface of the first section, and/or scales, the troughs and/or grooves and/or scales formed on the surface of the first section, whereby the edges of the troughs and/or grooves and/or scales are preferable again rounded. In particular, the edges have a radius of at least 0.2 mm, in particular from 0.2 mm up to 1 mm and in particular preferred from 0.5 mm and up. As a maximum depth for the troughs and/or grooves a depth between 2 mm and 4 mm proved as reasonable.

The length of the first section of the pin can be between 3 cm and 8 cm, whereby in an embodiment for use for patients with incontinence problems, the pin is usually designed longer (for example, between 5 cm and 8 cm) than for using the pin for patients without incontinence problems. The second section has preferably a length between 5 cm and 10 cm, for example, between 6 cm and 8 cm, whereby a good griping of the second section with the user's hand is made possible.

In a further embodiment of the pin, the first section has formed at the predetermined end a mushroom-shaped or spherical-shaped head on whose surfaces the receiving elements are provided, in particular, in the form of troughs. This embodiment is used, particularly, when only the outer anal region should be cleaned. Thereby, the maximum diameter of the mushroom-shaped head is selected such that an insertion of the head into the anal canal is not possible. Then, the maximum diameter should be, in particular, between 30 mm and 40 mm.

In a further embodiment of the pin, the second section is essentially configured cylindrically and has, in particular, a diameter of between 5 mm and 10 mm, whereby preferably a thickening with an enlarged diameter, compared to the rest of the cylinder, is provided and/or troughs on the surface of the second section are provided. Through the thickening and respectively the troughs, a tactile indication is provided for a user, such indication informs the user, at which position the pin is or how the pin is oriented, respectively. That embodiment is especially helpful if the pin is used by the patient himself, as the patient cannot see the pin in this case.

In a further embodiment of the pin, the second section of the pin extends from a second end, being opposed to the predetermined end of the pin, in its longitudinal direction. The pin includes, thus, the first section on the one side and the second section on the other side. In a further embodiment, there is further more the possibility that the pin has several and in particular two first sections, which extend from opposite, predetermined ends of the pin in its longitudinal direction, whereby the second section is provided between the first sections. In particular the second section extends itself thereby between two limiting elements. Thereby, a pin is provided that includes sections at both ends, which can be inserted into the anal region. Preferably, in that embodiment, two different first sections can be integrated in a single pin. In particular, a first section can have a maximum diameter at the one predetermined end such that it is insertable into the patient's anal canal being located behind the anus, whereas the first section at the other predetermined end has a maximum diameter, which is not insertable beyond the anus into the anal canal of a patient.

In a further embodiment, the pin includes one or more canals ending at the first section for supply of one or more substances, in particular of liquids (e.g., water) and/or oils, into the anal range of a patient. In particular, these substances can also include medically-active ingredients. Thereby, there is the possibility that the substances are supplied from the outside over a terminal at the pin to the one or more canals. Likewise there is the possibility that the one or more canals are connected with a reservoir integrated in the pin, from which the one or more substances are supplyable to the canals.

In some embodiments of the pin, a pressurizing device is provided that stands in interactive connection with the reservoir, over which pressurizing device by applying pressure by a user the substance or substances can be pressed in that or the canals. In that case, there is no need to provide a further device for supplying or receiving substances at the pin. In an embodiment, the pressurizing device includes a piston pressing onto the reservoir, whereby by immediate pressure onto the piston or by air pressure applied via a pressure bellow onto the piston, the substance or substances can be pressed in that or the canals. Where appropriate, one can also do it without such a piston, so that the reservoir is immediately connected with the pressure bellow for applying pressure. In a further embodiment, the reservoir can be formed from an elastic material, which can be deformed by a user by applying pressure, to press that or the substances in that or the canals. Thus, by the elastic reservoir itself, a pressurizing device can be provided.

The pin can be made of various types of materials. For example, the pin can consist at least partly of plastic and/or metal and/or compostable materials, in particular of Makrolon® and/or Elastollan® and/or Purell LDPE, HDPE and PP resins and/or cellulose and/or corn starch and/or aluminum and/or stainless steel.

Beside the above-described cleaning pin, any embodiment of the cleaning pin can be used in a method for hygienic and/or medical treatment of the anal region of a patient, whereby for performing the hygienic and/or medical treatment, the pin is moved within the anal region in such a manner that with the help of the receiving element or the receiving elements arranged on the surface of the first section, stool remainders are removed from the anal region and/or substances are supplied to the anal region.

The pin can be used for hygienic and/or medical use in the human anal region so that a soothing and efficient hygienic and/or medical treatment of the anal region can be performed. The pin cleans the outer anal region before the sphincter, so that soothingly and effectively stool remainders can be removed. Moreover, the pin provides a simple way, using suitable substances (or fluids), such as active agents, which can be brought into the anal region and, in particular, into the anal canal behind the sphincter.

### DRAWING DESCRIPTION

FIG. 1 shows a schematic representation of the fundamental structure of a pin in accordance with a first embodiment;

FIGS. 2-4 show various embodiments of basic forms for the first section of a pin;

FIGS. 5-7 show detail views of embodiments of various surface structures for the first section of a pin;

FIGS. 8-11 show various views of the first embodiment of a pin;

FIGS. 12-14 show various views of a second embodiment of a pin;

FIGS. 15-16 show various views of a third embodiment of the pin;

FIGS. 17-19 show various views of a fourth embodiment of a pin;

FIGS. 20-22 show various views of a fifth embodiment of the pin;

FIGS. 23-25 show various views of a sixth embodiment of the pin;

FIG. 26 shows a view of a seventh embodiment of the pin;

FIG. 27 shows a view of an eighth embodiment of the pin;

FIG. 28 shows a view of a ninth embodiment of the pin;

FIGS. 29 and 30 show various views of a tenth embodiment of the pin;

FIGS. 31-32B show various views of an eleventh embodiment of the pin;

FIGS. 33 and 34 show various views of a twelfth embodiment of the pin; and

FIGS. 35 and 36 show various views of a thirteenth embodiment of the pin.

In the accompanying figures, same or corresponding elements from different embodiments are referred to in a large extent with the same reference numerals.

### DESCRIPTION

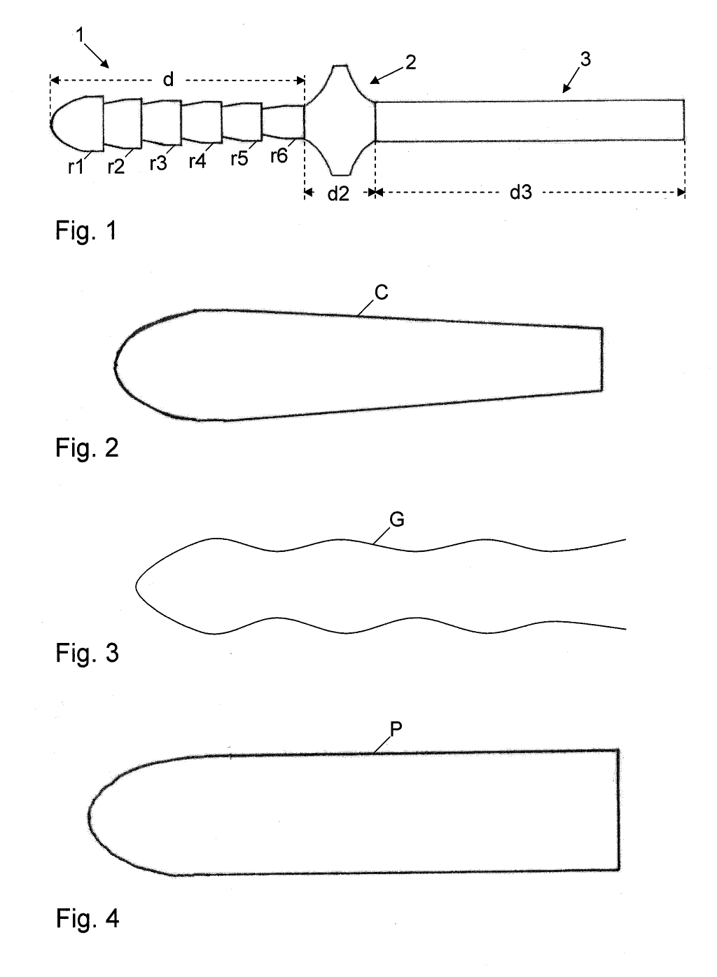

FIG. 1 shows schematically, in a side view, the structure of a first embodiment of a pin for hygienic and/or medical application. In the following, the pin is also referred to as a cleaning pin, independently of it being only usable for a medical or a hygienic or both applications. The pin includes a first section in the form of a cleaning portion 1, which is insertable into the anal region of a human patient. Next to the cleaning portion, there is a limiting element in the form of an intermediary part 2, which is annularly shaped and serves as a stop in the area of the anus, to prevent pushing the pin too far in into the anal canal. Next to intermediary part 2, there is a second section in the form of a tool holder 3, which can be held by a user, i.e., in particular, by the patient himself and/or, where appropriate, by a physician, to introduce the pin in the anal region and to accomplish the cleaning of the anal region described below.

The pin as shown in FIG. 1 is configured to be brought through the sphincter into the inner anal region, to clear the anal canal behind the anus from stool remainders. The cleaning portion 1 is associated with a length, which in FIG. 1 is referred to as d and is between 3 cm and 8 cm for performing an effective cleaning of the anal canal. For example, a length of 4 cm proved to be suitable. The length, referred to as d2, of the intermediary part 2 is within the range of 1 cm to 3 cm. The length, referred to as d3, of the tool holder 3 is selected such that it can be gripped well by a user and is, for example, between 5 cm and 10 cm, in particular, about 7 cm.

The intermediary part 2 as shown in FIG. 1 is positioned fixed in its axial position on the longitudinal axis of the pin. It can, where appropriate, be configured to be adjustably set in its axial position and, where appropriate, be also removable from the pin, for providing various sizes of intermediary parts on the pin. Where appropriate, also cleaning portion 1 can be releasably attached at the remainder of the pin, for example, screwed in, so that depending upon anatomical and/or pathological circumstances of the user, the cleaning portion is replaced by other cleaning portions.

As the pin is used in a sensitive body region, injuries within the anal range must be avoided by the pin. Therefore, at least for cleaning portion 1, types of material are used that do not have a casting seam and/or casting burr as well as having a roughness that is not too large. In particular, the median roughness should be within the range of 4 μm and less. To reach a simple and careful insertion of the pin into the anal region, the front end of the cleaning portion is rounded, whereby the curvature replicates the form of the slender side of a popsicle.

As shown in FIG. 1, cleaning portion 1 is configured in a step-shape, whereby each step is formed by a conical recess between two short cylindrical sections at the corresponding axial positions r1 to r6. With the help of these recesses, transporting stool remainders to the outside can be affected by withdrawing the first section out of the patient's anal canal, as is described below in more detail. The embodiment of FIG. 1 can be used in patients who have a normally closing sphincter and are without incontinence problems. For those patients, the maximum diameter of the cleaning portion, which in FIG. 1 is at the position r1, should not be selected to large. In particular, the maximum diameter should be between 8 mm and 20 mm, e.g. about 10 mm. The basic form of the first section 1 is conical, i.e., the cylindrical sections at the positions r1 to r6 decrease continuously in diameter. That conical surface form, which is formed by a covering surface around the cylindrical section of the pin, is again indicated in FIG. 2 and referred to with reference numeral C.

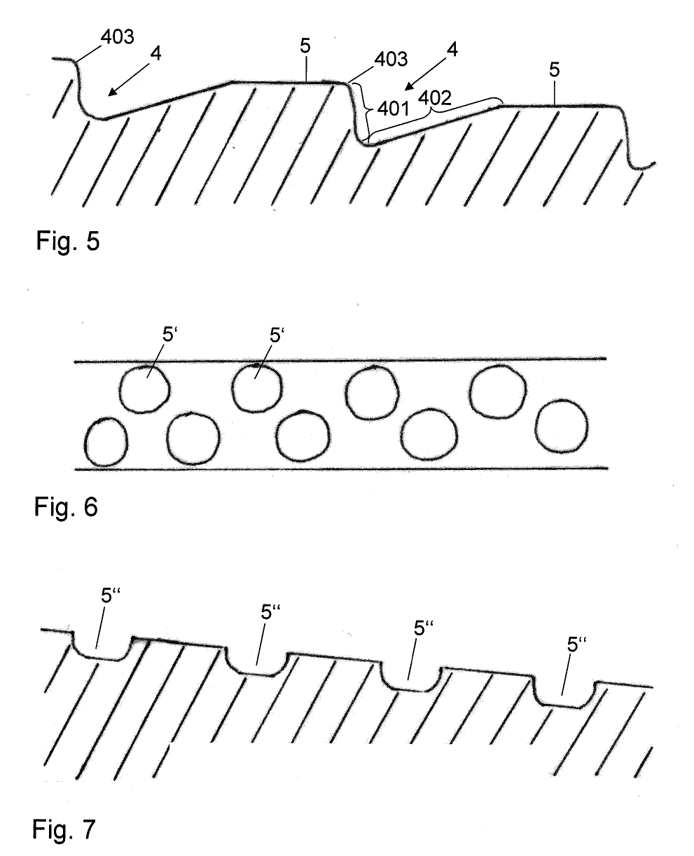

Instead of the conical basic form, the covering surface can be configured, where appropriate, in a wave-shape, whereby a wavy basic form is indicated with uniform waves in FIG. 3 and referred to with reference numeral G. Beside those basic forms, it is also possible to use, where appropriate, a parallel or cylindrical basic form, which is shown in FIG. 4 and referred to with reference numeral P. The conical basic form, which has the largest diameter at the tip of the cleaning portion and whose diameter is reduced in steps up to the intermediary part 2, is then of advantage, if mainly stool remainders need to be transported out of the anal canal. The parallel basic form is then of advantage if substances (like, e.g., active ingredients) need to be transported into the anal canal. The wave-shape basic form is then of advantage if both transporting stool remainders to the outside and also transporting substances to the inside needs to be equally achieved by the pin.

Independently of the represented basic forms, cleaning portion 1 needs surface structures, with which transporting stool remainders to the outside and/or transporting substances to the inside is enabled. In the example of FIG. 1, transporting stool remainders to the outside is in particular effected by the respective steps, which are again reproduced in an enlarged sectional view in FIG. 5. Each step is formed by an annular recess 4, which extends between two cylindrical sections referred to with reference numeral 5 in FIG. 5. In a modification, one can provide instead of or additionally to the annular recesses also helical recesses extending along the surface of the first section. The recess is formed by a steep falling edge in the form of a cut-in 401, whereby the depth of the cut-in is between 1 mm and 4 mm for achieving a good cleaning effect. Beginning from the deepest point of the recess 4, the recess extends then along a flat slope 402 up to the next cylindrical section 5. To avoid injuries of the mucosa in the anal canal and/or the sphincter, the edges 403 of each of the recesses 4 extending circumferentially around the cleaning portion are rounded, whereby a curvature diameter of between 0.2 mm and 1 mm, in particular, of 0.5 mm, has proven to be practical. In one embodiment, the length of a step from an edge 403 up to the next edge 403 is within the range of between 3 mm and 10 mm, in particular, about 6 mm.

As already apparent in FIG. 1, each of the radii of sections 5 decreases stepwise from the position r1 to the position r6, whereby the decrease of the radius can be chosen, for example, such that the diameter of the cleaning portion is tapered from r1 to r6 between 5% and 20%. It is also possible that the diameter decreases only once from the position r1 to the position r2 and then remains constant. In the embodiment shown in FIG. 1, however, the diameter of adjacent cylindrical sections 5 reduces by about 0.4 mm to 0.5 mm. In the embodiment of FIG. 1, altogether five steps are formed; however, the number of steps and also the depth of the steps (i.e., the cut-ins 401) can be varied in a suitable manner. In particular, the number of steps and/or the depth of the steps can be increased or decreased, to adapt cleaning portion 1 to the circumstances within the anal region of the patient and/or to the objective of the prophylactic application of the cleaning pin. Thereby, among other things, there are also shorter or longer and/or flatter or deeper alternative embodiments for the length and the depth of the individual steps in comparison with the cleaning shown in FIG. 1 or FIG. 5.

In the embodiment of FIG. 1, the step depth is constant from the first step at the front end of the first section to the last step. It is, however, also possible that the step depth varies, especially the step depth can decrease from maximum value at the tip of the cleaning portion to the intermediary part 2 continuously or decreasing after each second or third step, so that thereby the steps become ever more flat towards the intermediary part. For this embodiment, the particularly deep zones of the anal canal are cleaned. A more intensive cleaning in the region of the sphincter can be effected by an embodiment of the cleaning portion, for which the step depth increases from the tip to the intermediary part.

In the foregoing, an embodiment of step-shaped receiving elements was described, with which, in particular, stool remainders are removed from the anal canal behind the sphincter. Instead of such step-shaped receiving elements, also other types of receiving elements can be formed on the surface of the cleaning portion. For example, on the surface of the cleaning portion, receiving elements can be provided in the form of troughs, as indicated schematically in FIG. 6. That figure shows a section of the surface of a cleaning portion having a plurality of troughs, whereby some of the troughs exemplarily are referred to with reference numeral 5′. Likewise there is the possibility that grooves are formed on the surface of the cleaning portion that extend annularly or helically around the cleaning portion. Such grooves are shown in FIG. 7 and referred to with reference numerals 5″. FIG. 7 shows thereby a section of a cross sectional view of a cleaning portion with respective grooves. Instead of troughs or grooves, the surface of the cleaning portion can also be configured to be scaly, whereby likewise a removing of stool remainders is achieved by the intermediate spaces between scales being on top of each other.

Also for the embodiments with troughs and/or grooves and/or scales as receiving elements, it needs to be guaranteed that the mucosa in the patient's anal canal is not injured when using the cleaning pin. Accordingly, the edges of the troughs and/or grooves and/or scales are rounded analog to the edges of the step-shaped receiving elements in a suitable manner, in particular, with a radius of about 0.5 mm. Furthermore, it is possible that the various types of receiving elements according to FIG. 5 to FIG. 7 are combined with one another. For example, different types of receiving elements can be provided in a portion of the surface. Likewise, various types of receiving elements can also be, where appropriate, arranged within overlapping regions and/or several kinds of receiving elements can be mixed among themselves.

With the foregoing described types of receiving elements, stool remainders, like e.g. organic particles, bacteria, or other chemical compounds, can be caught in a simple manner from the surface of the mucosa of the anal canal behind the sphincter and, by withdrawing the cleaning portion, they can be transported to the outside. In this manner, a very good anal hygiene is achieved, and furthermore, and also a prophylactic effect is achieved, as is described below in more detail. In particular, also oils or medicaments can be introduced with the receiving elements, so that after the use of the pin, a fine film on the mucosa, which is based on an introduced oil or medicament, remains in place of the stool remainders that have been transported out.

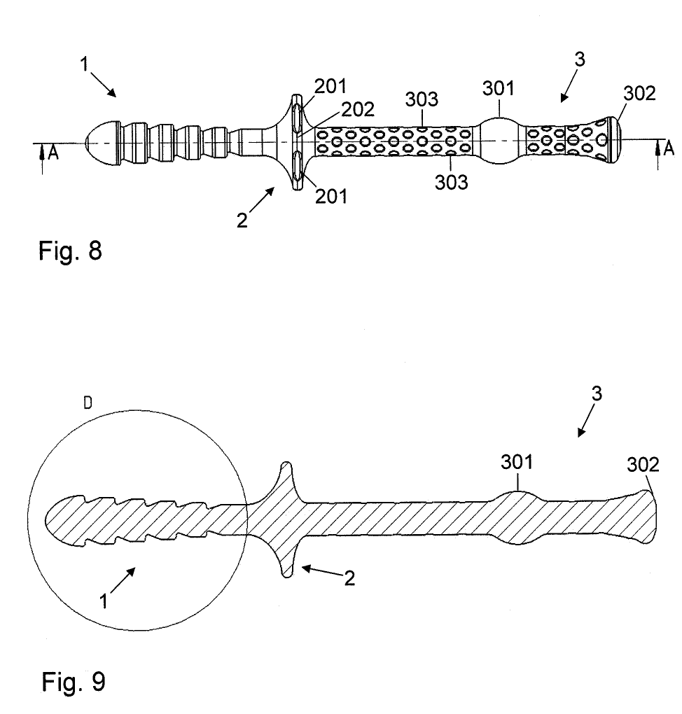

FIG. 8 shows a side view of a specific embodiment of the embodiment of a cleaning pin schematically shown in FIG. 1. One recognizes again cleaning portion 1, which is configured in a step-shaped manner in the same way as in FIG. 1, whereby now the surface structure of the individual steps is clearly evident. Furthermore, one recognizes that intermediary part 2 is configured plate-like, whereby the edge of the plate is formed with a plurality of flats 201 and rounded regions 202 in between. This becomes also apparent from the cross sectional view of the plate of FIG. 8 that is shown in FIG. 11. One recognizes in FIG. 11 that six flats 201 with rounded regions 202 located in between them are formed at the edge of the plate. Flats 201 have a length d4 of approximately 7.5 mm. The distance d5 between two opposite flats 201 is approximately 22 mm. The distance d6 between two opposite rounded regions 202 is approximately 23 mm.

The embodiment of the plate according to FIG. 11 is only exemplarily and one can also apply arbitrary other forms of a plate, in particular, circular, elliptical, rectangular, squared, or other polygon shaped forms. Important is only that the size of the plate is such that a penetration of the pin beyond the plate into the anal canal is prevented. That is, the plate size is that large that the cross section of the plate cannot penetrate over the sphincter into the anal canal. The plate diameter should be accordingly in a range between 25 mm and 40 mm, for example, in a range between 25 mm and 35 mm.

Tool holder 3 of the embodiment of the cleaning pin of FIG. 8 is configured essentially in a cylindrical manner, includes, however, in the rear region a thickened portion 301 and comprises at its rear end a gate 302. With the help of thickening 301, the user of the pin is provided with a tactile region, in which position and/or orientation the pin is currently. This is in particularly favorable when the cleaning pin is used by the patient himself, because the patient cannot see the pin during cleaning. Moreover, the surface of the tool holder comprises a plurality of troughs, only some of which are referred to with reference numeral 303. Also those troughs provide a tactile feedback of the position of the pin to the user.

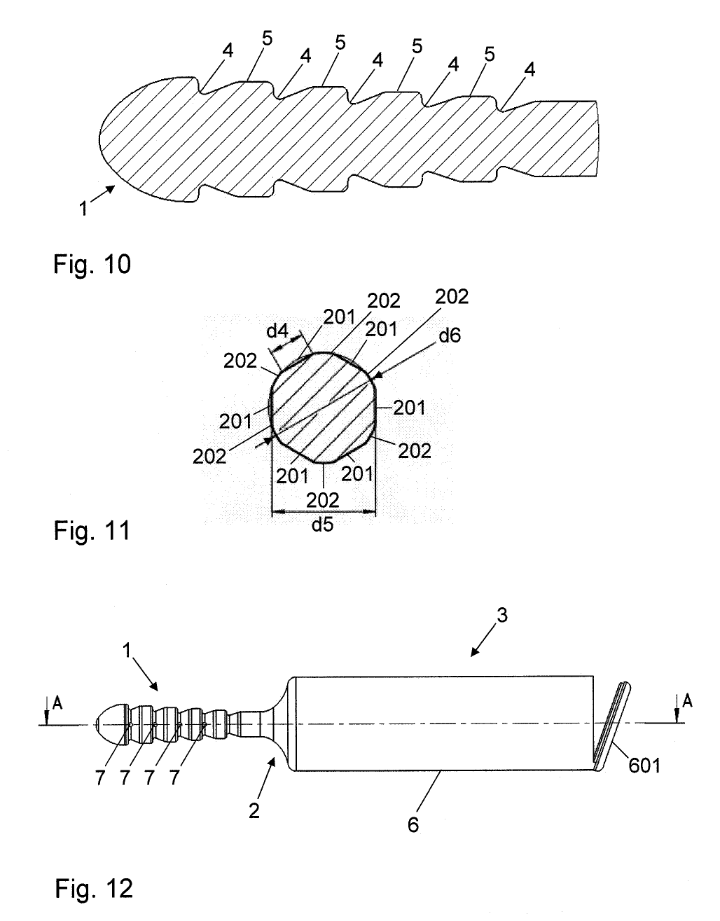

FIG. 9 shows a sectional view of the cleaning pin of FIG. 8 along the line A-A. In FIG. 9, one sees that the pin is made of solid material, whereby materials for the pin can include one or more of stainless steel, aluminum, or plastic. FIG. 10 shows again an enlarged view of the detail D of FIG. 9. One sees in FIG. 10 again clearly the profile of individual step-shaped recesses 4. In particular, it becomes apparent that in this embodiment all recesses 4 have the same dimensions, whereby, however, the radius of cylindrical sections 5 in between recesses 4 continuously decreases. Furthermore, the egg-shaped head of the cleaning portion is clearly apparent.

The cleaning procedure of the anal region for the above described embodiment of a cleaning pin is performed, in particular, in such a manner that the end of cleaning portion 1 and, where appropriate, also the individual steps are wetted with a few drops of a sliding-promoting (lubricious) liquid, such as, e.g., natural oils or paraffin oil or liquid Vaseline. Then the end of the cleaning portion is set on the anus and inserted with smooth pressure through the closing muscle of the sphincter into the inner anal region. Subsequently, the pin is moved back and forth carefully along its longitudinal direction, until the pin is removed from the anal region. By moving back and forth, the present stool remainders are collected from the anal canal behind the sphincter within the steps and are transported to the outside. Finally, the cleaning portion is cleaned, e.g., rinsed below running, preferably warm water. The used oils reduce a strong adhesion of the stool remainders, which accordingly can be washed off easily.

The above described cleaning procedure of the inner anal region can be repeated once or several times. By the surface structure of recesses 4 with cut-in 401 and slopes 402 as well as by a suitable choice of the diameter of cylindrical sections 5, an easy gliding of cleaning portion 1 into the internal anal area becomes possible without resistance. When withdrawing the cleaning portion from the anal canal, the stool remainders are picked up by the annular steps, whereby an injury of the mucosa of the anal canal is avoided by the rounded edges of the steps. The same effect can also be achieved by the described above alternative surface structures, i.e., by grooves and/or troughs and/or scales.

The use of the cleaning pin in combination with oil is, e.g., suggested with an inflammatory provoked situation of the anal region or when beginning with this hygiene method. Preferably, the oil is then applied both on the tip of cleaning portion 1 and on the individual steps. An essential part of the so applied oil remains after the cleaning procedure with a maintaining effect within the anal canal. In addition, one can pad with the oil-wetted round end of the cleaning portion over the anus, in order to moisten it in the outer region with some of the applied oil. This enables then an easy insertion of the cleaning device into the anal canal.

Generally, a wetting with oil or alternative substances has a plurality of functions. The wetting simplifies penetration into the anal canal, effects careful cleaning procedure, a careful sliding-out, and reduces adhesion of stool remainders at the cleaning portion for an easier intermediate and final cleaning of the cleaning portion. Furthermore, such a wetting can exercise a maintaining and anti-inflammatory effect of the tissue in the anal canal. Moreover, the penetration of particular therapeutic effective compositions is made possible. The application of the cleaning pin with oils can thus be combined with medical effective compositions. An example is the treatment of inflammations and/or vessel extensions, like, e.g., with hemorrhoids. For this application, the cleaning pin is wetted with an oil, which includes one ore more medical active ingredients. The cleaning pin is used as usual, only that when withdrawing the pin the oil with the medically-active ingredients remains at least partly in the anal canal and deploys its effect therein. For an application of the cleaning pin, also a wetting of the cleaning portion with water may be sufficient.

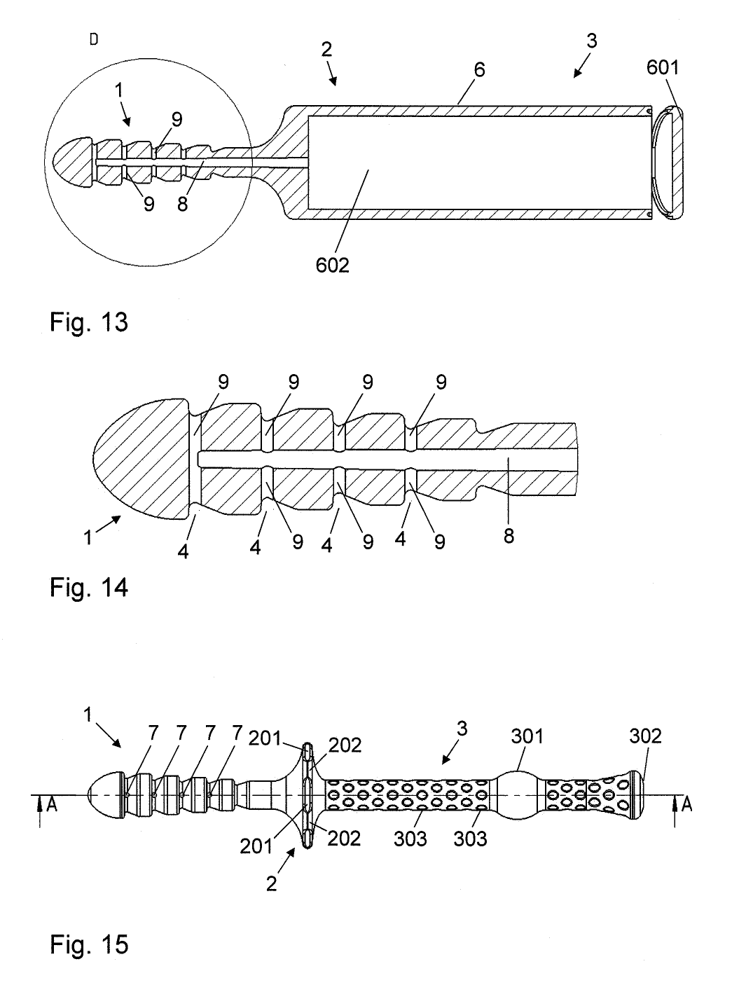

In the following, various modified embodiments of a cleaning pin are described. In a side view, FIG. 12 shows a cleaning pin with a fixedly connected water reservoir 6. In its form, cleaning portion 1 is configured in an analog manner with respect to the cleaning portion according to FIG. 8. Cleaning portion 1 follows again an intermediary part 2, which now, however, forms a conical transition element towards cylindrical reservoir 6, which is sealed at its rear end by a lid 601. The reservoir is filled via the lid with water (where appropriate also with oils), whereby the water reaches via canals inside cleaning portion 1 corresponding outlets 7 arranged at the deepest point of the individual recesses. FIG. 13 shows a sectional view along the line A-A of the cleaning pin shown in FIG. 12. One recognizes that reservoir 6 comprises a cylindrical hollow interior 602, which is connected over a main canal 8 with respective side canals, only some of which are referred to with reference numeral 9. The first four steps of the cleaning portion are provided each with two side canals, respectively, which extend into opposite directions out of main canal 8 and end at respective outlets 7.

In the embodiments of FIG. 12 and FIG. 13, reservoir 6 has also the function of tool holder 3, whereby the reservoir is formed from an elastic material such that cavity 602 of the reservoir can be made smaller by pressing onto the material. The material is configured such that the cavity returns into its original cylindrical shape after the pressure application. When using the cleaning pin, the reservoir is filled with water and by pressing onto the reservoir, water is forced via canals 8 and 9 out of outlets 7, so that at the time of performing a cleaning procedure, an additional cleaning of the anal canal by water is done. FIG. 14 shows the detail D of cleaning portion 1 shown in FIG. 13. One recognizes again the penetration of the cleaning portion with water canals, whereby it, in particular, is apparent that main canal 8 has a somewhat larger diameter than side canals 9. Moreover, it is clear that all side canals 9 are arranged such that they lead to outlets, each of which is at the deepest point of individual recesses 4.

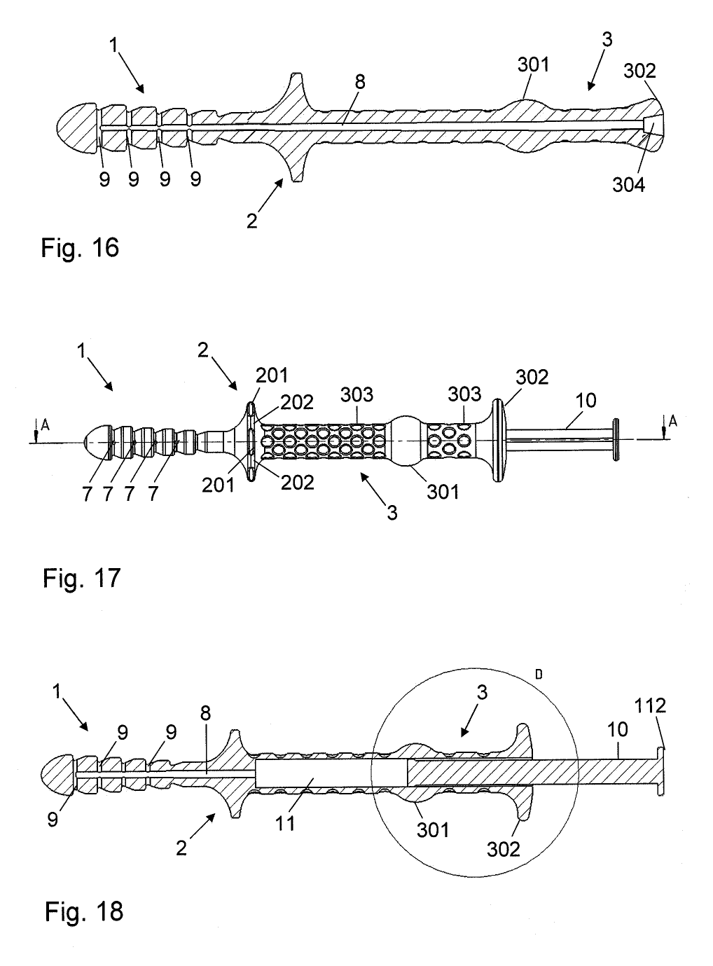

FIG. 15 shows a third embodiment of a cleaning pin. The cleaning pin is thereby configured essentially analogue to the cleaning pin of FIG. 8, whereby the pin is, however, in this case formed not from solid material, but a canal system is provided within the pin, which ends again in corresponding outlets 7 at the deepest points of the individual recesses. The canal system is apparent in FIG. 16, which shows the cross section of the pin of FIG. 15 along the line A-A. One recognizes that central canal 8, which extends in the embodiment of FIG. 12 to FIG. 14 only up to a reservoir 6, runs in this case through entire tool holder 3 up to the rear end at gate 302. At gate 302, a terminal 304 is provided, which forms the connection to main canal 8 and thus to the internal canal system. That embodiment allows, e.g., flushing with water over a tube, which is connected to terminal 304 and through which water is supplied to the canal system, the water being discharged at outlets from cleaning portion 1 again through corresponding side canals 9. The structure of the canals inside cleaning portion 1 and the form of the cleaning portion are identical to the embodiment shown in FIG. 12 and FIG. 14.

FIG. 17 shows a further embodiment of a cleaning pin. The structure of cleaning portion 1 as well as intermediary part 2 corresponds thereby to the embodiment of FIG. 15. Tool holder 3 differs, however, from the embodiment of FIG. 15 therein that it has a larger diameter and forms a piston system in a syringe type. To this, a manually operated piston 10 is provided, that is inserted via the rear end of tool holder 3 into a corresponding cavity. This is repeatedly shown in the sectional view of FIG. 18, which shows the cross section of the cleaning pin of FIG. 17 along the lines A-A. In FIG. 18 one recognizes that inside the tool holder a cylindrical cavity 11 is formed, along which piston 10 is guided. Cavity 11 can thereby again be filled with water and/or therapeutic effective liquids, whereby by pressing on the end face 112 of the piston, cavity 11 is made smaller and thereby liquid is pressed into a corresponding canal system with canals 8 and 9. Analog to the embodiments of the FIG. 12 to FIG. 16, the liquid is dispensed via outlets 7 from the cleaning portion.

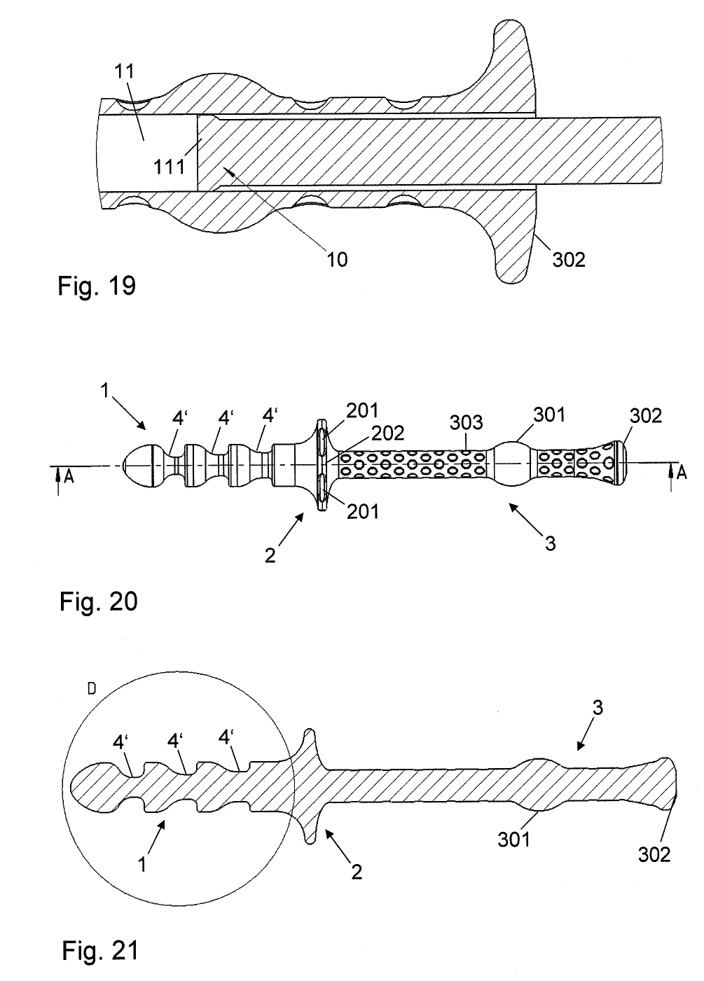

In the embodiment of FIG. 17 and FIG. 18, the cleaning of the anal canal is supported by pressurizing a piston with additional water or corresponding liquids. In contrast to the embodiment of FIG. 15 and FIG. 16, the back sided gate 302 is configured in an enlarged manner as a plate and forms thereby an abutment for operating piston 10 as a syringe. FIG. 19 shows once more the enlarged view of detail D of FIG. 18. One recognizes that piston 10 has a diameter at its front end 111, which is configured to fit flush within the inner diameter of cavity 11. Where appropriate, an annular seal can be provided at piston end 111 for sealing the cavity. Next to end 111, there is a piston portion with a diameter being reduced with respect to the inner diameter of the cavity, which does not rub to the inner wall of the cavity. Thereby, it is ensured that a frictional force is acting at the inner cylindrical surface of cavity 11 only at the front end of the piston, such that the friction is reduced and the piston operation is made easier.

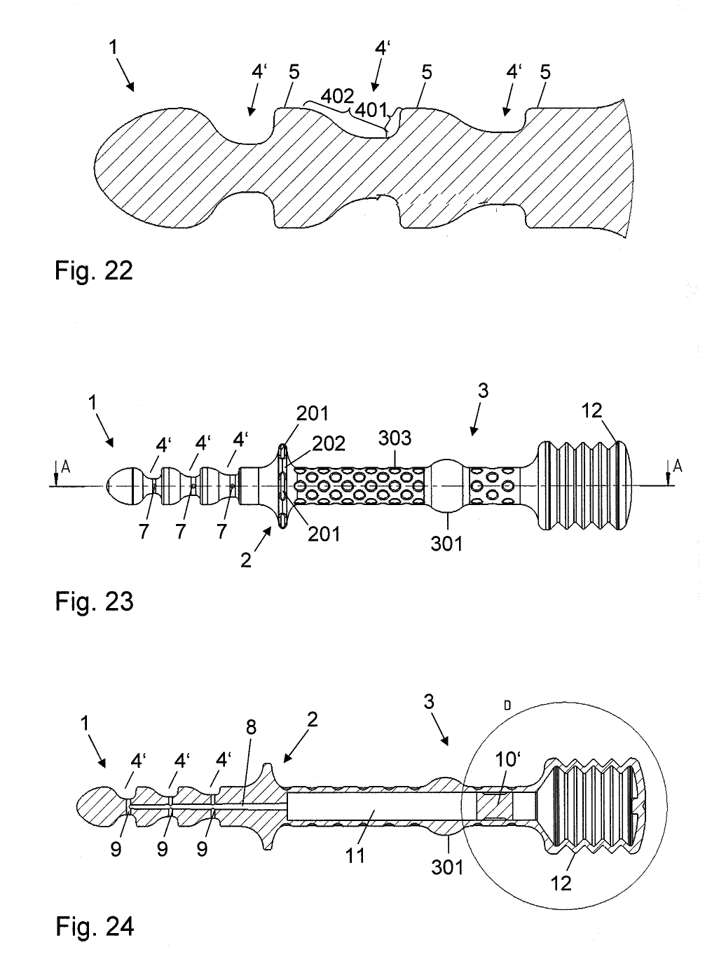

FIG. 20 shows a fifth embodiment of the cleaning pin. It relates to a medical embodiment of the cleaning pin, which serves primarily for applying medically-active ingredients behind the sphincter into the anal canal. In the embodiment of FIG. 20, the structure of intermediary part 2 and tool holder 3 does not differ from the structure of the embodiment of FIG. 8. The substantial difference between the embodiment of FIG. 20 and the preceding embodiments is in the structure of the cleaning portion 1, which has now the primary function of the transport of active ingredients into the anal region and, therefore, can be appropriately referred to as transportation portion. Analogous to the embodiment of FIG. 8, cleaning portion 1 is configured step-shaped, whereby the recesses of the individual steps, referred to in FIG. 20 with 4′, in this case, however, have the reverse form as in the embodiment of FIG. 8. That is, the recesses extend away from the tip of cleaning portion 1 first along a flat slope and rise then steeply at a cut-in. This becomes apparent once more in the sectional views of FIG. 21 and FIG. 22. FIG. 21 shows thereby the cross sectional view along the line A-A of FIG. 20. In FIG. 21, one recognizes, in particular, that the cleaning portion is made integral from one piece without a cavity, as it is the case also in the embodiment of the cleaning pin of FIG. 8.

FIG. 22 shows once again a view of detail D of FIG. 21. One recognizes clearly that the recesses are configured as transportation bags with a curved form, which is implemented more than in the embodiment of FIG. 8. In particular, flat slope 402 of FIG. 22 is in a slightly S-shaped wave form, whereas corresponding slopes 402 of FIG. 5 extend essentially straight. A further difference to the embodiment of FIG. 8 consists of the fact that cylindrical sections 5 located between the pockets 4′ have in this case a constant diameter and do not decrease in their diameter in direction away from the tip of the cleaning portion 1. Accordingly, the cleaning portion of FIG. 21 and FIG. 22 is an implementation of a cylindrical basic form according to FIG. 4. Besides this basic form, preferably also the use of a curved basic form in accordance with FIG. 3 may be applicable. In a simple manner, active ingredients can be supplied into the anal canal via the pockets of FIG. 22 and a cylindrical or curved basic form. Due to the form of the pockets, the active ingredients, which can be an oil or liquid introduced into the pockets, can be well introduced into the anal canal and remain to a large extent in the anal canal.

FIG. 23 shows a sixth embodiment of a cleaning pin. In its structure, the cleaning pin corresponds to a large extent to the cleaning pin of FIG. 20. In contrast to FIG. 20, in this case there is, however, again provided an internal canal system ending at outlets 7 and a corresponding cavity for receiving water and/or active ingredients, as shown in the embodiment of FIG. 18. In contrast to FIG. 18, there is, however, provided a pressure bellow 12 at the rear end of tool holder 3, by which respective air pressure can be applied onto reservoir 11 within tool holder, to press oil with a therein contained active ingredient out of the canal system.

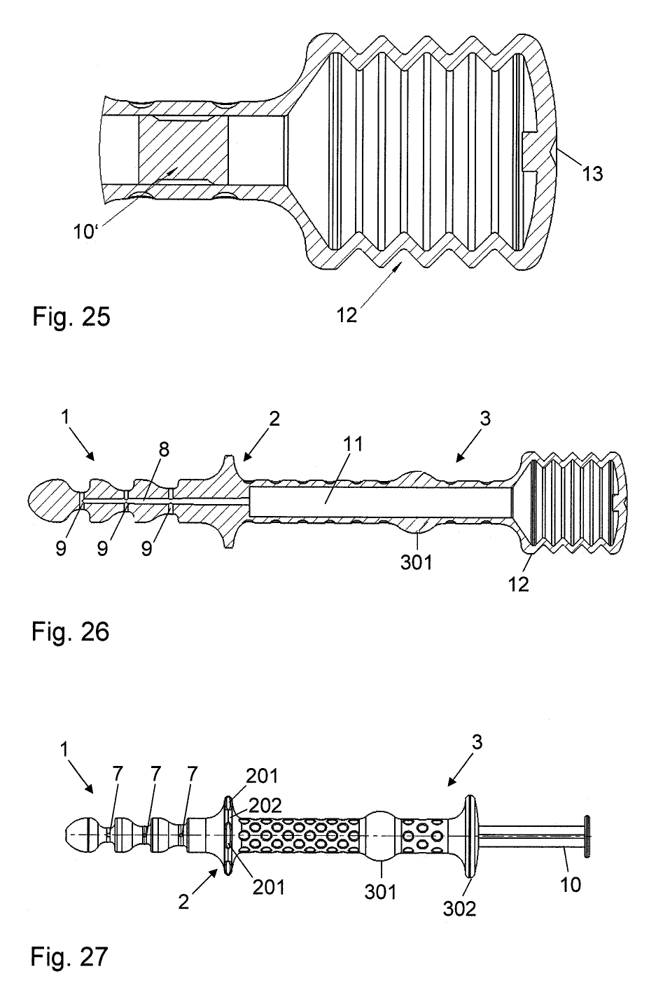

FIG. 24 shows the cross section along the lines A-A of FIG. 23 and in that figure, one recognizes central canal 8 as well as side canals 9 that are configured analog to the embodiment of FIG. 18. Likewise one sees reservoir 11 of the active substance, which is also present in the embodiment of FIG. 18. Instead of a prolonged piston 10 in the manner of a syringe, as shown in FIG. 18, in this case, however, a small cylindrical piston 10′ is used, which points with one of its sides to pressure bellow 12. By seaming of the pressure bellow, a user can exercise an air pressure onto piston 10′, so that the piston presses via the air pressure onto active substance reservoir 11 and thereby reduces it in size, whereby liquid with the corresponding active ingredient is discharged from canals 8 and 9. FIG. 25 shows once more the view of detail D of FIG. 24. One recognizes, in particular, that a valve 13 is provided at the rear end of pressure bellow 12, by which the seaming and the re-expanding of pressure bellow 12 is made possible. Furthermore, the accurate structure of the piston 10′ is made once more apparent, which has a diameter corresponding to the inner diameter of the cavity 11 at its two ends. Between the two ends, the piston comprises thereby a portion with a reduced diameter, whereby the friction of the piston is reduced.

As a seventh embodiment, FIG. 26 shows a modification of the embodiment of FIG. 23 to FIG. 25. The only difference of this embodiment in comparison with the embodiment of FIG. 22 to FIG. 25 consists of the fact that the air pressure of pressure bellow 12 presses in this case directly without the use of a piston onto reservoir 11 having the active ingredient contained therein. As an eighth embodiment, FIG. 27 shows a further modification of the embodiment of FIG. 23 to FIG. 25. In contrast to the embodiment of FIG. 23 to FIG. 25, in this case once more a prolonged piston 10 of the type of a syringe instead of pressure bellow 12 is used for pressing the active ingredient out of reservoir 11. To that extent, the structure of tool holder 3 corresponds to the tool holder of the embodiment of FIG. 17 to FIG. 19, so that it is referred to the corresponding description of FIG. 17 to FIG. 19.

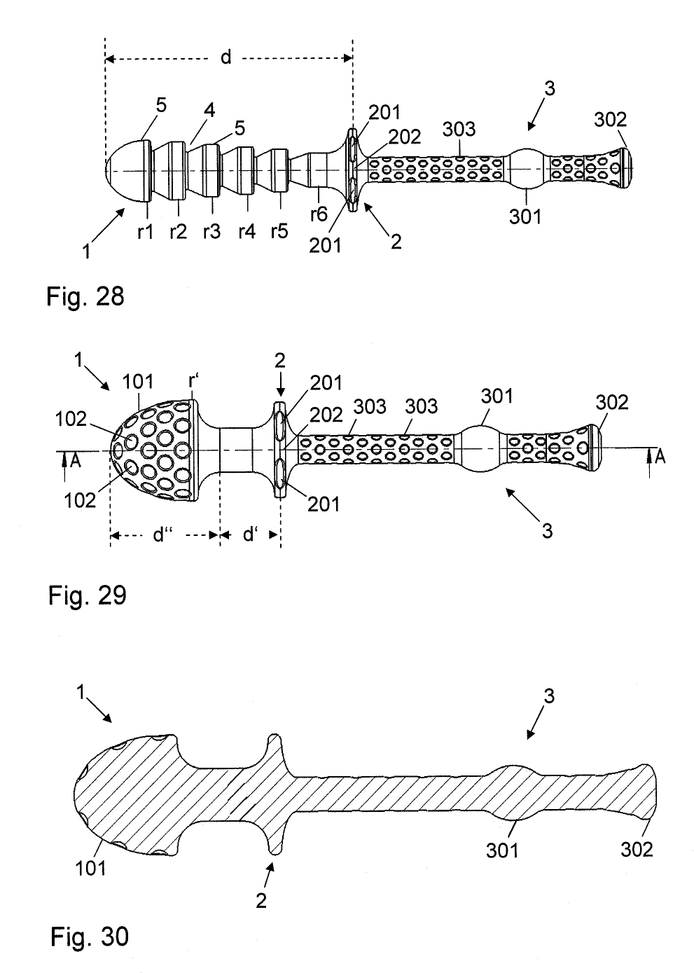

FIG. 28 shows a ninth embodiment of the cleaning pin. Therein, the cleaning pin is configured in its molding analogously to the cleaning pin of FIG. 8. In particular, cleaning portion 1 also includes respective recesses and cylindrical sections, which for clarity reasons are referred to only partly with reference numerals 4 and 5. Likewise the tip of the cleaning portion is rounded. The difference of the embodiment of FIG. 28 with respect to the embodiment of FIG. 8 consists of the fact that cleaning portion 1 has a larger diameter. In particular, the maximum diameter at the axial position r1 is larger than in the embodiment of FIG. 8 and is in particular at least 15 mm. The decrease of the diameters at the axial positions r1 to r6 is once again continuous, whereby the reduction of the diameter from an axial position to the next is between 0.5 mm and 2 mm. Due to the enlarged cleaning portion, individual recesses 4 can be configured with a bigger depth. In particular, they have a depth of between 2 mm and 5 mm. Likewise the length D of the cleaning portion is longer than in the embodiment of FIG. 8 and is between 6 cm and 8 cm (e.g. 7 cm). The embodiment of FIG. 28 is intended for the use for incontinent patients with a leaking sphincter. By enlarged cleaning portion 1, the incontinent user having the leaking sphincter is able to clean the anal canal as far as possible. Thus, an excretion of larger amounts of stool to the outside is reduced.

FIG. 29 shows a tenth embodiment of a cleaning pin, which is intended for cleaning only the outside anal region before the sphincter. Intermediary part 2 and tool holder 3 are therein configured analog to the embodiment of FIG. 8, the pin, however, has a different cleaning portion 1 with a mushroom-shaped or strawberry-shaped head 101, which begins in a distance d′ of about 2 cm to 3 cm behind intermediary part 2. In a modification, the head can also have the form of a ball. The length d″ of the head in axial direction is preferably thereby between 2 cm and 4 cm. The head is characterized in that it has a maximum diameter at the axial position r′ that is larger than the maximum diameter of the cleaning portions described above. In particular, the diameter is between 25 mm and 40 mm, preferably it is larger than 30 mm. The diameter is chosen such that a penetration of the cleaning portion is no longer possible through the sphincter into the anal canal. By the distinct curvature and a soft embodiment of head 101, cleaning the anus can be performed after bowel movement, without a penetration of the tip into the anal canal behind the anus being possible.

For loosening and removing stool remainders, a plurality of troughs is provided on the surface of head 101, which for clarity reasons are only partially provided with reference numerals 102. For cleaning, head 101 is wetted with water and, by moving the head over the anus, stool remainders are collected in troughs 102, whereby the pin can be repeatedly immersed in water during the use, where appropriate. The cleaning procedure can take place under simultaneous sprinkling with water, where appropriate. Analog to the preceding embodiments, the edges of the troughs are again rounded, respectively, to avoid injuries in the anal region. The troughs are configured similarly to a golf ball, however, being deeper. Their depth is, for example, between 0.3 mm and 1 mm. Therefore, the troughs can, on the other hand, bind water applied for the cleaning and, on the other hand, enable a smooth cleaning of the outside anal region, without the user bringing its fingers in direct contact with the stool. That embodiment serves, in particular, to replace the finger cleaning of the anus still being conventional in some cultures with cleaning with a cleaning pin after the bowel movement.

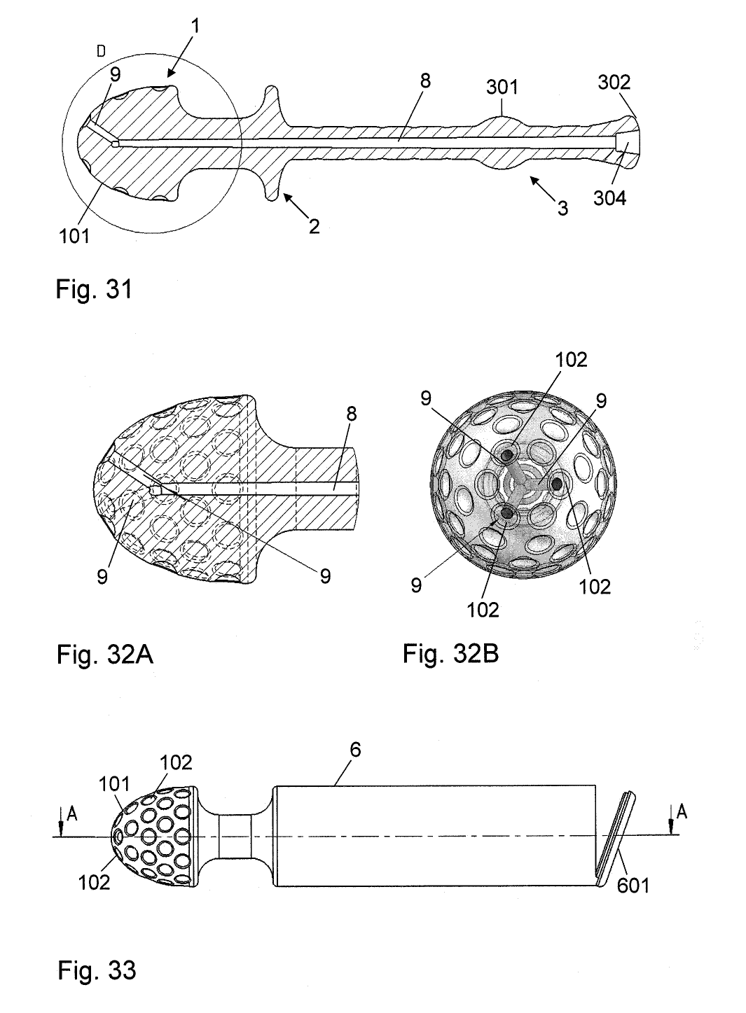

FIG. 30 shows a sectional view along the line A-A of FIG. 29. One recognizes that the cleaning pin is made of a solid material. There is, however, also the possibility to provide canals in the cleaning pin for guiding liquids to the head 101, as it was the case already also in some of the preceding embodiments. As eleventh embodiment, such an embodiment of a cleaning pin is shown in FIG. 31 in a cross sectional view. The outside structure of the cleaning pin corresponds therein to the cleaning pin of FIG. 29. In analogy to the embodiment of FIG. 15 and FIG. 16, canals 8 and 9 extend in this case inside the pin, whereby main canal 8 extends from a terminal 304 at the rear end of tool holder 3 in axial direction into head 101 and branches there into several side canals 9, whereby in FIG. 31 only one of the side canals is visible. The side canals open up finally at the surface of the head. As before, a tube can be fixed to terminal 304, which is connected to a water supply, to provide during the cleaning water to head 101 and, thus, to the anal region to be cleaned, whereby a particularly intense cleaning of the outside anal region is achieved.

FIG. 32A and FIG. 32B show once again in a sectional view and in a plain view the front of the structure of cleaning head 101 in accordance with the detail D of FIG. 31. One recognizes, in particular, that three side canals 9 are provided, which similar to a tripod branch from the main canal 8 in the front portion of head 101 and end in outlets, which each are positioned at the bottom of a corresponding trough 102. When using a plurality of such side canals, the cleaning effect within the anal region is improved having a corresponding supply of water.

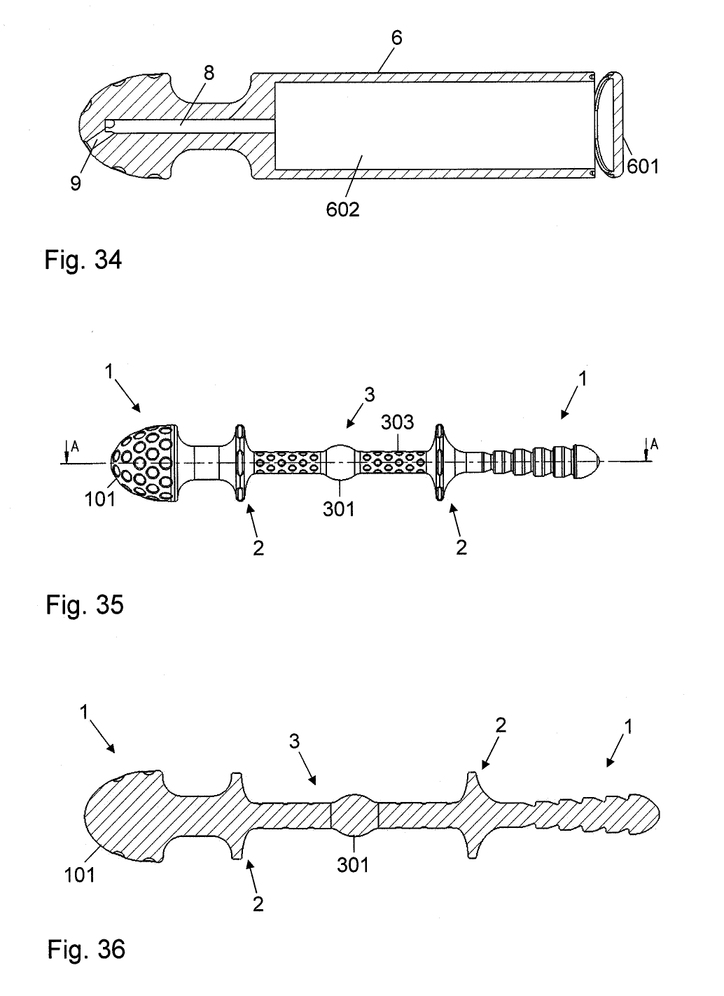

As twelfth embodiment, FIG. 33 shows as a side view a modification of the embodiment of the cleaning pin of FIG. 31. In this embodiment, analog to the embodiment of FIG. 12 to FIG. 14, a water reservoir 6 is provided, which can be filled via a resealable lid 601. The outer walls of the reservoir are configured in this case again to be slightly elastic, so that by means of employing pressure by a user water can be supplied to a main canal and respective side canals in head 101 of the cleaning portion. The internal structure of the pin of FIG. 33 is once again apparent from a cut view along the line A-A which is shown in FIG. 34. One recognizes, thereby, once again that reservoir 6 is formed of a cylindrical cavity 602, from the front end of which a main canal 8 extends, which branches into side canals 9 in analogy to the embodiment of FIG. 31. For the use of the cleaning pin in accordance with FIG. 33 and FIG. 34, lid 601 is opened and water reservoir 6 is filled with water. Then the cleaning tip 101 is put onto the outside anal region. With slightly moving back and forth and some pressure onto the water reservoir, the outside anal region is then cleaned mechanically and under the addition of water.

FIG. 35 shows a thirteenth embodiment of a cleaning pin. In contrast to the preceding embodiments, the pin can in this case be used for cleaning the inner anal canal and also only the cleaning of the outside anal region. Therefore, corresponding cleaning portions 1 are provided at both ends of the cleaning pin, whereby the left cleaning portion provides head 101 in accordance with the embodiment of FIG. 29 and FIG. 30 and the right cleaning portion is configured as a step-shaped cleaning portion in accordance with the embodiment of FIG. 8. Both cleaning portions once again are limited by corresponding intermediary parts 3, to prevent a too far insertion of the cleaning portions into the anal region. In contrast to the preceding embodiments, tool holder 3 is in this case centrally arranged between two intermediary parts 2. It exhibits again a thickening 301, which is in this case centrally arranged between two intermediary parts 2 and also serves to provide a tactile region to the user the position and/or orientation of the cleaning pin.

The cleaning pin of FIG. 35 has the advantage that it can be used depending upon need only for the cleaning of the outside anal region after the bowel movement or a more intensive cleaning of the anal canal behind the anus. The embodiment of FIG. 35 is thereby formed of solid material, as can be seen in the cross sectional view along the line A-A, which is shown in FIG. 36. Where appropriate, however, it is also possible to provide canals and/or reservoirs in analogy to the corresponding embodiments described above, to enable the supply of liquids and/or active ingredients when using the cleaning portion.

Preferred materials for the preparation of the cleaning pin in accordance with the embodiments described above are high-quality, break-proof, and elastic plastics. Also metals can be used. For medical applications, the material should allow disinfection and sterilization at increased temperature, e.g., up to 121° C. For the one-time use, less high-quality plastics or materials decomposing after the cleaning rapidly in water can be used. In particular, compostable substances can be used for the preparation of the cleaning pin, such as, e.g., corn starch or pulp.

Particularly suitable materials for the cleaning pin are hard-elastic plastics, such as, e.g., the plastic Elastollan® or Makrolon® and/or hard rubber and comparable materials. In their handling in the sensitive anal region, theses slightly elastic materials have the advantage of adapting better and an even more preserving cleaning of the mucosal surface in the anal canal. A special coating of the surface of the cleaning portion of the pin is of advantage for better and more careful sliding and also for a still better cleanability of the pin. As coating material, e.g., Teflon can be used. Furthermore, beside coarse surface structures in the form of receiving elements, also micro structured surfaces can support a better bonding of stool remainders and, thus, an efficient transport of those remainders to the outside. The cleaning portion of the cleaning pin should be configured lightly colored, so that stool remainders can be recognized easily during intermediate and final cleaning of the anal region. In particular, light opaque white colors, like, e.g., RAL 9003 signal-white, should be used as a color for the cleaning portion. Where appropriate, the pin can also be configured to be fully transparent, so that it appears glass-like.

The cleaning pin can also provide a medical effect in addition its cleaning effect. In particular, the use of the pin can be prophylactic for a plurality of diseases within the anal region. By use of the cleaning pin in the anal canal behind the sphincter, a so far not existing form of the prophylaxis against arising diseases of the anal canal that is pointing into the future is achieved. The entire inflammation tendency of the anal region can be positively affected by the use of the cleaning pin and the removal of partially aggressive stool remainders accomplished thereby. The vessel extensions conditional to other causes, e.g., with hemorrhoids, or also other diseases of the anal region have the chance to heal and calm down faster by the removal of the stool remainders. Injuries of the anal canal, such as, e.g., fissures, do not develop severely and can heal more rapidly. Blockages of glands, skin folds, and crypts are reduced and thereby the inflammation of these regions is reduced. The passage of stool remainders to the outside onto the outside skin and, thus, the inflammatory change of that outside skin are minimized. Thus, the application of the pin represents a completely new hygienic concept, which extends beyond the prior, to the patients' recommended hygiene practices in the anal region, because those always stop before the sphincter.

The above described cleaning pins permit a diverse range of applications. The cleaning and care of the anal canal with the cleaning pin direct after the bowel movement is the standard application. Here, the newly generated stool remainders are mostly immediately removed and can no longer unfold their adverse influence in this region. When applying corresponding oils onto the cleaning pin, a maintaining oil film is furthermore left in the anal canal. When used regularly, the most complaints in the anal region are prophylactically repressed. Also when not regularly applied, it can be reasonable, to perform this cleaning of the anal canal with the cleaning pin where appropriate, for example, when irritations and itching occurs, because, e.g., owing to circumstances, one was not able to directly apply after the bowel movement this cleaning regularly. Also with that procedure, very positive prophylactic effects in that body region can be achieved. With slight complaints within the anal region, such as, e.g., itching or other forms of uncomfortable feelings, a prophylactic application of the cleaning device is likewise reasonable because in most cases this slight complaint disappears immediately and does not come back with regular, prophylactic application any longer.

The above described cleaning pins can be used in a supportive manner also with the therapy of diverse medical indications within the anal region. Stool remainders contain in addition to organic food remainders and a large amount of bacteria, also a various amount of chemical compounds from the digesting process which can be of a very different composition depending on the flow of the digesting process. In medicine, these stool remainders are considered as far as possible as a physical substrate, normal for the body that is compatible and unproblematic. However, this is only correct if one assumes a healthy condition of the tissue of the anal canal.

The healing promoting effects of the cleaning pin regarding numerous disease forms of the anal canal are based on the subsequently explained factors. In the case of an inflammatory change of the body tissue, it comes to the excretion of mediators of the body (messenger agents), which attract immune cells and in addition, increase the permeability of vessel walls and the mucous membrane. Additionally, in regards to the inflammatory response, the cell coherency (cooperation of the cells) can be destroyed and thus the natural barrier function of the mucous membrane can be affected. Therefore, bacteria and toxins from the stool can penetrate easier into the pathological and inflammatory modified tissue of the anal canal. The conventional protective mechanisms, by which the mucous membrane in the anal canal in a healthy state usually protects itself from the stool and its ingredients, are thus affected. The body's immunologic and reparative mechanisms cannot control the inflammatory process any longer and a rapid healing is prevented. Pathological processes of the mucous membrane in the anal canal, as they occur, for example, for hemorrhoids, anal fistulas, anal fissure, and other infections of the anal canal arise, cause a change of the permeability of the vessels and the mucosa of the anal canal, and it comes to a negative interaction between the stool remainders (organic food remainders, bacteria, numerous various chemical compounds as residues from the digesting process) and the pathological modified tissue. Also for the external skin outside of the anal canal, a regular contact with stool remainders leads in the medium-term to an inflammatory change of this skin region and, in the extreme case, to eczema.

In the following, it is explained, for which diseases in the anal canal the cleaning pin deploys a medical effect. An application is the use of the cleaning pin with hemorrhoids. Hemorrhoids are an arteriovenous annular vessel pad system at the transition from the rectum to the anus and serve to fine seal the anus. One speaks of a hemorrhoids disease or hemorrhoids, if an enlarged or lower moved hemorrhoidal pad causes complaints. Thus, bleeding and/or itching, burning, wetting, and stool smearing within the anal region can occur. The causes of the hemorrhoids disease are not clarified completely. As a cause, stool masses or an inflammation can prevent the drain from the vascular pads within the transition zone and thus lead to a magnification. Hemorrhoids can be impacted by digestion of food with less fiber, the erected walk, long seating, genetic disposition (weakness of connective tissue) as well as strong and prolonged pressing during the bowel movement. In addition, the releasing of a too soft unformed stool, e.g., after an abuse of laxatives, can lead to hemorrhoids. The pathological processes of the hemorrhoids disease lead finally to a modification of the anal mucous membrane in the form of fibrosis (modification of the connective tissue), ulcerations (ulcer formations), and fissures (mucous membrane-lacerations). Thus, there is more often a negative interaction between the stool remainders (organic food remainders, bacteria, numerous various chemical compounds as residues from the digesting process) and the pathological modified hemorrhoids pad. Therefore, it can be assumed that an essential removal of the stool remainders from the anal canal and the introduction of maintaining anti-inflammatory substances, such as, e.g., oils, with the help of the cleaning pin lead to a significant improvement of hemorrhoid complaints.

The cleaning pin can also be used for the treatment of and/or prophylaxis of abscesses. Abscesses and thereof resulting fistulas within the anal region develop on the basis of proctodeal glands from a cryptitis (inflammation of pockets and/or glands in the anal canal). The mucous membrane bags at the boundary of external skin and transition epithelium of the anal canal for the mucosa of the rectum (rectum) serves as an entrance gate for the pathogens. This boundary resembles a jagged line. One calls the jags anal papillae, and the “valleys” between the jags crypts, which frequently correspond to probable pockets and within their depth the exit canals of anal glands end. Clogs the exit canal of such a gland, it results in the above mentioned cryptitis. As the cleaning pin leads to a significant reduction of the stool remainders in the anal canal, and at the same time can leave maintaining and anti-inflammatory substances, the risk of a blockage of the anal glands becomes significantly reduced and the tendency for inflammation of these structures in the course of a cryptitis is reduced.

The cleaning pin can also be used for the treatment of and/or prophylaxis of an anal eczema. Anal eczema is one of the most frequently occurring proctologic diseases. It is characterized by a long lasting, itching, and inflammatory modification of the external skin around the anus. It is not a disease per se, but an accompaniment of different dermatological, allergological, microbiological, or proctologic processes. There is evidence that, in particular, this disease is maintained active by leaking stool remainders, but at least is promoted thereby. The partially aggressive stool remainders provoke the external skin and worsen an existing eczema or lead for sensitive skin also directly to anal eczema. The cleaning pin strongly limits the chance for leaking stool remainders. This results in a faster healing of the external skin and in disappearing of the eczema. To the itching within the anal region, the same applies as for the anal eczema.

A further field of application is the use of the cleaning pin with fecal incontinence. Fecal incontinence means the loss of control over gas, liquid, or solid stool. One of the causes for this is the weakness of the sphincter. For lighter forms of this disease, it is helpful for the patient to clean the anal canal as far as possible after the dispensing of stool to limit a too rapid leaking of stool remainders through the weak sphincter. The cleaning pin is here a suitable tool, to provide more hygiene, security, and ease to those patients.

The cleaning pin serves also for the treatment of and/or prophylaxis of anal cramps, which are caused by pathological modifications in the anal region, such as, e.g., hemorrhoids, anal fissures, etc. The pathological modifications are accompanied by intense pain, which again causes a cramping of the closing musculature, which then leads to new, more violent pain, whereby anal cramps are caused. The cramping tissue is not as well supplied with blood and, therefore, does not recover as well. Thereof chronic fissures can result, which lead to the respective anal cramp or a stenosis (constriction). Thus, each measure, which reduced the inflammation tendency of the anal canal, is a helpful means against painful anal cramps. Regularly removing stool remainders from the anal canal after the bowel movement with the cleaning pin leads, therefore, to a significant reduction of those cramp-like complaints of the anal canal.

A further field of application of the cleaning pin is the treatment of and/or prophylaxis of thrombosis of anal veins. This relates to suddenly arising, usually hurting, nodes at the anus. The swelling is caused by a blood clot in a surface vein at the anus. It is frequently mistaken for hemorrhoids. As most frequent triggers for an anal thrombosis one considers the subjection to of coldness, physical exercise-, increased intra-abdominal pressure when coughing, lifting and/or pressing, for example, during bowel movement, a pregnancy and a birth, an unfavorable diet with too much alcohol and hot spiced food, and preceding proctologic surgery. It is assumed that the presence of large hemorrhoids favors the formation of anal thrombosis. A thrombosis (local blood thickening) blocks the vein mesh and initiates a local inflammatory response. Such inflammatory processes of the anal mucous membrane are again amplified by the negative interaction between the stool remainders and the inflammatory modified tissue. A regular removal of stool remainders from the anal canal with the cleaning pin leads to a significantly faster healing of these complaints and prevents an anew occurrence of the anal vein thrombosis to the greatest possible extent.

The cleaning pin can also be used when mariscae are present. Mariscae are skin folds, which are regarded as remnants of a healed anal thrombosis. In the case of this pathological modification of the anal canal, the stool remainders settle between the skin folds and cause an initiation and a development of an inflammation. The removal of the stool remainders with the help of the cleaning pin out of the anal canal leads to a significant improvement for those complaints in the anal canal.

The cleaning pin can also be used for the treatment of and/or prophylaxis of anal fissures. Anal fissures are mucous membrane-tearings, which, for example, are caused by pressing to hardly during bowel movement and a stool being very hard. Moreover, the tearings can develop from frequent inflammations due to a loss of elasticity of the anal skin. Diarrhea can also damage the sensitive skin of the anal canal that far that smallest impulses are sufficient for the development of an anal fissure. If an anal fissure is present, then the partially aggressive stool remainders settle into those mucous membrane wounds and hinder the healing process. The connection between stool remainders and this specific type of disease is here particularly significant. Regularly removing stool remains from the anal canal after the bowel movement and introducing the healing promoting oils and substances with the cleaning pin results in a significantly faster healing of those complaints and prevents to the greatest possible extent an anew occurrence of anal fissures and the other above described diseases of the anal canal.

1. A pin for hygienic and/or medical use in the anal region, comprising:
a first section extending from a predetermined rounded end of the pin in the pin's longitudinal direction and configured to be introducible into the anal region of a patient for performing a hygienic and/or medical application; and
a second section offset in longitudinal direction from the first section and configured to enable a user to hold the pin to perform the hygienic and/or medical application,
wherein one or more receiving elements are formed on the surface of the first section and are configured for removing stool remainders from the anal region.

2. The pin of claim 1, wherein at least one limiting element is provided between the first and second section, limiting the insertion of the pin to a maximum depth in the anal region.

3-4. (canceled)

5. The pin of claim 1, wherein the first section has a maximum diameter such that it is insertable into the patient's anal canal located behind the anus.

6. The pin of claim 1, wherein the first section has a maximum diameter such that it is not insertable into the patient's anal canal behind the anus.

7. The pin of claim 1, wherein the surface of the first section has a basic shape being rotationally symmetric with respect to the longitudinal direction of the pin.

8-9. (canceled)

10. A pin for hygienic and/or medical use in the anal region, comprising:
a first section extending from a predetermined rounded end of the pin in the pin's longitudinal direction and configured to be introducible into the anal region of a patient for performing a hygienic and/or medical application; and
a second section offset in longitudinal direction from the first section and configured to enable a user to hold the pin for performing the hygienic and/or medical application,
wherein one or more receiving elements are formed on the surface of the first section,
wherein the receiving element or the receiving elements comprise one or more steps, wherein a respective step is formed as an annular recess, extending in circumferential direction around the pin, or as a helical recess extending along the surface of the first section.

11. The pin of claim 10, wherein the length of the recess in longitudinal direction of the pin is between 2 and 10 mm and the maximum depth of the recess is between 1 and 5 mm.

12. The pin of claim 10, wherein the recess is rounded at its edges, whereby the edges have a radius of at least 0.2 mm.

13. The pin of claim 10, wherein the recess is shaped to have in its cross section a cut-in at an axial position and a rising slope extending in a direction away from or towards the predetermined end of the pin from the cut-in to an adjacent axial position.

14. (canceled)

15. The pin of claim 10, wherein, in a direction away from the predetermined end, the diameter of the first section decreases, or increases, or remains constant at axial positions.

16. (canceled)

17. The pin of claim 1, wherein the receiving elements comprise a plurality of one or more troughs, grooves, and scales, wherein the plurality of the one or more troughs, the grooves, and the scales are formed on the surface of the first section, and wherein the edges of the plurality of the one or more troughs, grooves, and scales are rounded and have a radius of at least 0.2 mm.

18. The pin of claim 17, wherein the troughs and/or grooves have a maximum depth of between 2 and 4 mm.

19. The pin of claim 1, wherein the first section has a length of between 3 and 8 cm and/or the second section has a length of between 5 cm and 10 cm.

20. The pin of claim 6, wherein the first section comprises a mushroom-shaped or spherical-shaped head formed at the predetermined end, on whose surfaces the receiving elements are provided.

21-22. (canceled)

23. The pin of claim 1, wherein the pin comprises a first section and a second section, each of which extend from opposite, predetermined ends of the pin in its longitudinal direction,
wherein the second section is provided between the first sections and wherein the first section at the predetermined end has a maximum diameter such that it is insertable into the patient's anal canal being located behind the anus, and the second first section at the other predetermined end has a maximum diameter such that it is not insertable beyond the anus into the patient's anal canal.

24-25. (canceled)

26. The pin of claim 1, further comprising:
one or more canals ending at the first section for supply of one or more substances in the patient's anal region.

27. (canceled)

28. The pin of claim 26, further comprising:
a reservoir integrated in the pin; and
a pressurizing device,
wherein the one or more canals are connected with the reservoir, via which the one or more substances are supplyable to the canals and the pressurizing device is in interactive connection with the reservoir and is configured for pressing the substances in the canals by the application of pressure by a user.

29-32. (canceled)

33. The pin of claim 1, wherein the pin is made at least partly of one or more of plastic, metal, and compostable material.

34. A method of providing prophylaxis for or supporting the therapy of medical indications within a patient's anal region with a cleaning pin comprising a first section and a second section, the first section having at least one receiving element arranged on its surface and the second section being configured as a tool holder, the method comprising:
holding the cleaning pin at the second section;
inserting the first section into the patient's anal canal behind the sphincter;
moving the pin within the anal canal in such a manner that stool remainders within the anal canal are caught by the receiving element or the receiving elements; and
removing the first section from the anal canal, thereby removing the stool remainders from the anal canal.

35. The method of claim 34, wherein the cleaning of the patient's anal canal causes a prophylactic and/or healing promoting effect for medical indication selected from the group consisting of hemorrhoids, anal abscesses, anal fistulas, anal fissures, cryptitis, anal eczema, fecal incontinence, anal cramps, thrombosis of anal veins, mariscae, and generally infections of the anal canal.

36. The pin of claim 1, wherein the surface of the first section is conical, cylindrical, or wavy.

37. The pin of claim 5, wherein the maximum diameter of the first section is between 8 and 25 mm.

38. The pin of claim 6, wherein the maximum diameter of the first section is at least 30 mm.

39. The pin of claim 10, wherein the recess is arranged between two adjacent axial positions and the axial position is arranged on a cylindrical partial section of the first section.

40. The pin of claim 17, wherein the grooves extend annularly or helically at the surface of the first section.

41. The pin of claim 26, wherein the substances include one or more liquids and oils.

42. The pin of claim 33, wherein the plastic includes one or more of Makrolon®, Elastollan®, and Purell resins; the compostable material includes one or more of cellulose and corn starch; and the metal includes one or more of aluminum and stainless steel.

43. A pin for hygienic and/or medical use in the anal region, the pin comprising:
a first section extending from a predetermined rounded end of the pin in the pin's longitudinal direction and configured to be introducible into the anal region of a patient for performing a hygienic and/or medical application; and
a second section offset in longitudinal direction from the first section and configured to enable a user to hold the pin to perform the hygienic and/or medical application;
wherein:
one or more receiving elements are formed on the surface of the first section and are configured for supplying substances to the anal region and the one or more receiving elements comprise one or more steps;
a respective step is formed as an annular recess, extending in circumferential direction around the pin, or as a helical recess extending along the surface of the first section;
the recess is arranged between two adjacent axial positions, where an axial position is arranged on a cylindrical partial section of the first section; and
the recess is shaped to have in its cross section a cut-in at an axial position and a rising slope extending towards the predetermined end of the pin from the cut-in to an adjacent axial position.
